(19) United States
(12) Patent Application Publication
(10) Pub. No.: US 2026/0207912 A1
(43) Pub. Date: Jul. 23, 2026

(54) MEDICAL INSTRUMENT AND MEDICAL DEVICE

(71) Applicant: FURUKAWA ELECTRIC CO., LTD., Tokyo (JP)

(72) Inventors: Yoshiki NOMURA, Tokyo (JP); Junichi HASEGAWA, Tokyo (JP)

(21) Appl. No.: 19/140,367

(22) PCT Filed: Dec. 22, 2023
(86) PCT No.: PCT/JP2023/046130

§ 371 Date: Jun. 18, 2025

(30) Foreign Application Priority Data
Dec. 23, 2022 (JP) 2022-206943

(51) Int. Cl. A61M 39/02; H02J 50/00; H02J 50/10; H02J 50/90; H02J 105/46

(52) U.S. Cl. CPC A61M 39/0208; H02J 50/005; H02J 50/10; H02J 50/90; A61M 2039/0238; A61M 2205/587; H02J 2105/46

(57) ABSTRACT

Provided are a medical instrument and a medical device with which it is possible to reduce the size of a body section by minimizing the space required for installation of a power-receiving coil. A medical instrument 10 that is to be implanted in vivo comprises: a body section 11 that has a concave part 11a open at the top and extending in the thickness direction, and that is capable of storing liquid in the concave part 11a; a lid part 12 that is made of a soft member which is penetrable by a needle and that closes the opening in the concave part 11a; a power-receiving part 13 having a power-receiving coil 13b for receiving power sent from a power-transmitting coil 22a; and a reporting part 14 that is connected to the power-receiving part 13 and that reports, by the power received by the power-receiving coil 13b, information relating to the position of the lid part 12. The power-receiving part 13 has a circuit board 13a whereon a plurality of conductor layers 13a2 are formed in the thickness direction. The power-receiving coil 13b is configured on the circuit board 13a by connecting the plurality of conductor layers 13a2 of the circuit board 13a to each other.

### TECHNICAL FIELD

The present invention relates to a medical device and a medical instrument that is implanted in a living body.

### BACKGROUND ART

A conventionally known medical instrument to be implanted in a living body is provided with a main body that has a recessed section extending in the thickness direction and having an upper surface with an opening and that is capable of storing a liquid in the recessed section, a lid part that is formed from a soft member through which an injection needle can be inserted and that closes the opening of the recessed section of the main body, a power reception coil that receives power sent from a power transmission coil, and a circuit board that is provided with a light-emitting unit for emitting light by means of the power received by the power reception coil and is disposed in the main body at a position on the outer circumference side of the recessed section (see, for example, Patent Document 1).

Bringing an outside-of-living-body unit having the power transmission coil close to the medical instrument implanted in a living body causes power to be transmitted from the power transmission coil to the power reception coil and thus causes the light-emitting unit to emit light, so that the position of the lid part can be identified outside the living body by means of the light emitted from the light-emitting unit.

### CITATION LIST

### Patent Document

Patent Document 1: PCT International Publication No. WO2022/102393

### DISCLOSURE OF THE INVENTION

### Problems to Be Solved by the Invention

In the medical instrument noted above, the light-emitting unit is installed on the upper surface side of the circuit board extending along the outer circumference side of the recessed section of the main body, and the power reception coil is disposed on the lower surface side of the circuit board. The power reception coil is formed by winding a conducting wire having a prescribed wire diameter, so the main body will be large-sized in the thickness direction in a case where the power reception coil is sequentially wound in the thickness direction of the main body, and the main body will be large-sized in the radial direction in a case where the power reception coil is sequentially wound in the radial direction of the main body. Meanwhile, the main body of the medical instrument to be implanted in a living body needs to have a reduced radial size in order to reduce the invasiveness in the living body.

An object of the present invention is to provide a medical instrument and a medical device that allow a main body to have a reduced size owing to a reduced installation space for a power reception coil.

### Means for Solving the Problems

A medical instrument according to the present invention is implanted in a living body and includes a main body that has a recessed section extending in the thickness direction and having an upper surface with an opening and that is capable of storing a liquid in the recessed section, a lid part that is formed from a soft member through which an injection needle can be inserted and that closes the opening of the recessed section, a power reception unit that has a power reception coil for receiving power sent from a power transmission coil, and an informing unit that is connected to the power reception unit and informs information pertaining to the position of the lid part by means of the power received by the power reception coil, wherein the power reception unit has a circuit board in which a plurality of conductor layers are formed in the thickness direction, and the power reception coil is formed in the circuit board by connecting the plurality of conductor layers of the circuit board to each other.

In the medical instrument according to the present invention, the circuit board is formed in a ring shape surrounding the outer circumference side of the recessed section of the main body.

The medical instrument according to the present invention further includes a positioning structure that positions the circuit board with respect to the main body.

In the medical instrument according to the present invention, the number of windings of the power reception coil is two or greater per one conductor layer of the circuit board.

A medical device according to the present invention includes an outside-of-living-body unit that has a power transmission coil for sending power and a medical instrument that has a power reception coil for receiving the power sent from the outside-of-living-body unit and that is to be implanted in a living body, wherein the medical instrument includes a main body that has a recessed section extending in the thickness direction and having an upper surface with an opening and that is capable of storing a liquid in the recessed section, a lid part that is formed from a soft member through which an injection needle can be inserted and that closes the opening of the recessed section, a power reception unit that has the power reception coil, and an informing unit that is connected to the power reception unit and informs information pertaining to the position of the lid part by means of the power received by the power reception coil, the power reception unit has a circuit board in which a plurality of conductor layers are formed in the thickness direction, and the power reception coil is formed in the circuit board by connecting the plurality of conductor layers of the circuit board to each other.

### Effects of the Invention

According to the present invention, the power reception coil is formed in the circuit board, thereby allowing the installation space for the power reception coil to be reduced in comparison to when the power reception coil is formed by winding a conducting wire, so that the main body can have a reduced size.

### BRIEF DESCRIPTION OF THE DRAWINGS

FIG. 1 is a cross-sectional side view of a medical device according to one embodiment of the present invention;

FIG. 2 is a perspective view of a medical instrument according to one embodiment of the present invention;

FIG. 3 is an exploded perspective view of a medical instrument according to one embodiment of the present invention;

FIG. 4 is a cross-sectional view of a principal portion of a circuit board according to one embodiment of the present invention;

FIG. 5 is a cross-sectional plan view of a circuit board according to one embodiment of the present invention;

FIG. 6 is a cross-sectional side view of a medical device according to another embodiment of the present invention;

FIG. 7 is a circuit diagram illustrating an example of the power reception circuit of the medical instrument of the present invention;

FIG. 8 is a circuit diagram illustrating another example of the power reception circuit of the medical instrument of the present invention;

FIG. 9 is a circuit diagram illustrating another example of the power reception circuit of the medical instrument of the present invention; and

FIG. 10 is a circuit diagram illustrating another example of the power reception circuit of the medical instrument of the present invention.

### PREFERRED MODE FOR CARRYING OUT THE INVENTION

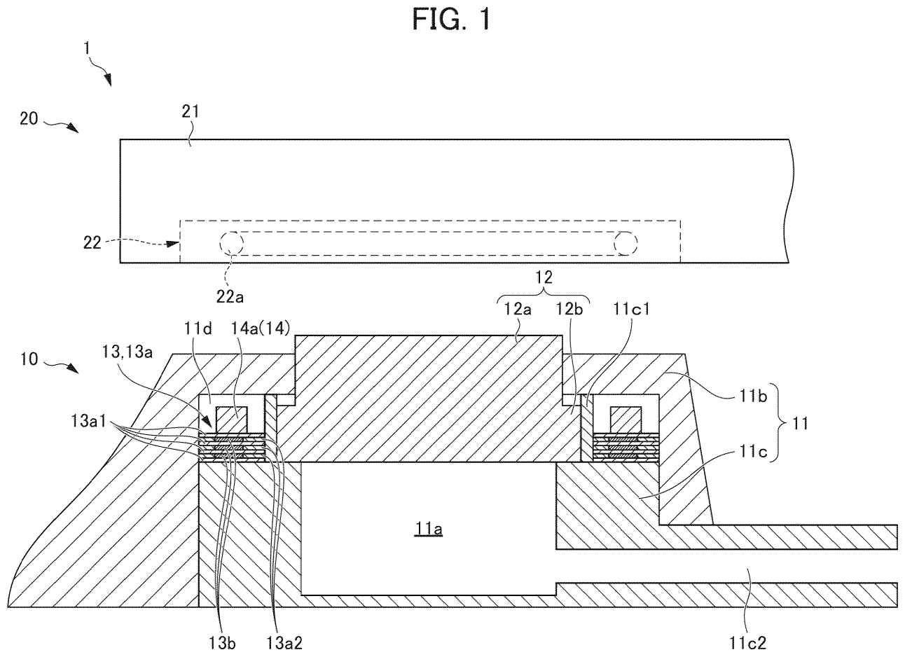

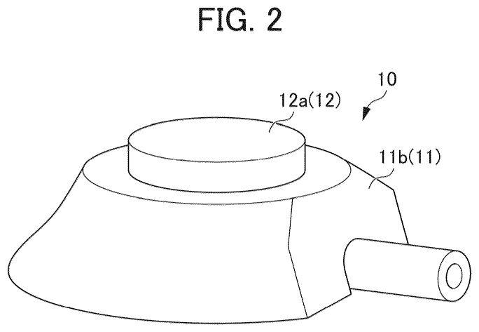

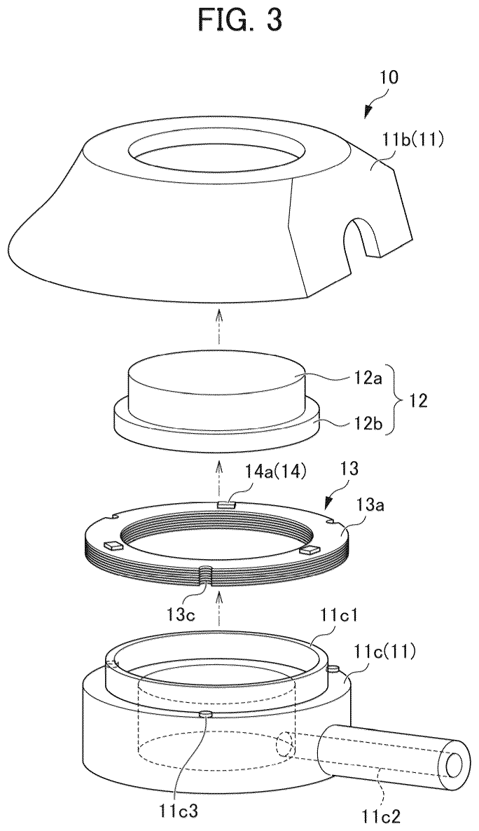

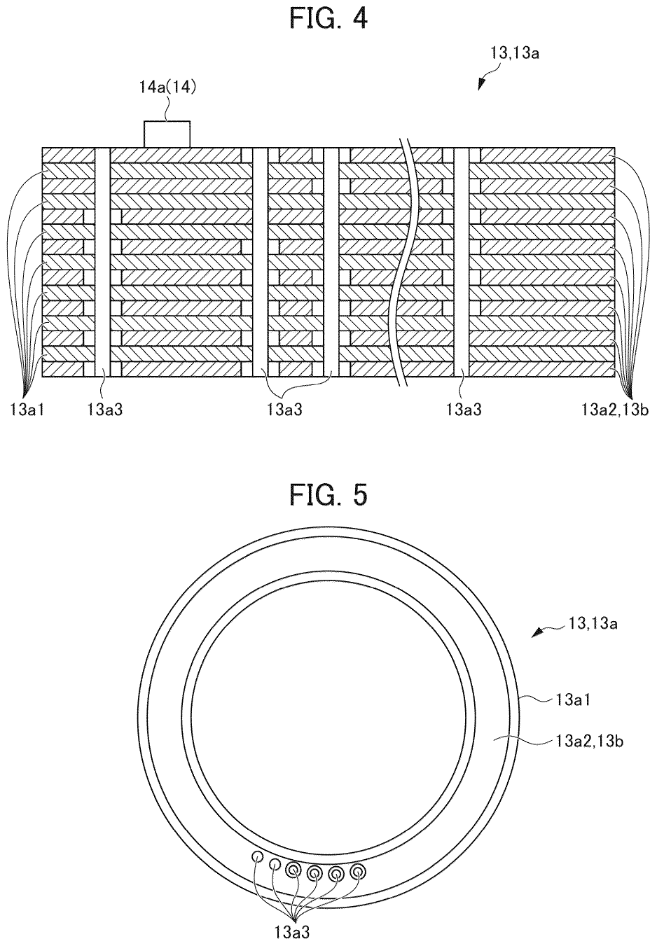

FIGS. 1 to 4 depict one embodiment of the present invention. FIG. 1 is a cross-sectional side view of the medical device. FIG. 2 is a perspective view of the medical instrument. FIG. 3 is an exploded perspective view of the medical instrument. FIG. 4 is a cross-sectional view of a principal portion of the circuit board. FIG. 5 is a cross-sectional plan view of the circuit board.

As depicted in FIG. 1, a medical device 1 of the present embodiment includes a medical instrument 10 that is used in a state of being implanted in a living body such as a human or an animal, and an outside-of-living-body unit 20 for supplying power to the medical instrument 10. The medical device 1 supplies power from the outside-of-living-body unit 20 to the medical instrument 10 in a noncontact manner.

The medical instrument 10 is a subcutaneous port (CV port), i.e., a type of central vein catheter, for injecting a medical solution into a central vein located in the vicinity of the heart of a living body. As depicted in FIGS. 1 to 3, the medical instrument 10 includes: a main body 11 that has a recessed section 11a in which a liquid such as a medical solution can be stored; a lid part 12 that closes the opening of the recessed section 11a of the main body 11; a power reception unit 13 that receives power sent from the outside-of-living-body unit 20; an informing unit 14 that is connected to the power reception unit 13 and informs information pertaining to the position of the lid part 12 by means of the power received by the power reception unit 13; and a catheter (not illustrated) that has one end in communication with the recessed section 11a of the main body 11, and another end side to be inserted into a central vein of the living body.

For example, the main body 11 is formed from a resin material such as polyphenylene sulfide (PPS), polyethersulfone (PES), or polyetheretherketone (PEEK) and is formed in a substantially truncated cone shape. The main body 11 has formed therein the recessed section 11a, which has an upper surface with an opening and extends straight toward the bottom surface side. A liquid is stored in the recessed section 11a. The main body 11 is formed from an upper side member 11b and a lower side member 11c. The power reception unit 13 and the informing unit 14 are accommodated in a space 11d formed between the upper side member 11b and the lower side member 11c. The space 11d is formed in a ring shape so as to surround the outer circumference side of the recessed section 11a. A partition wall 11c1 to serve, in the space 11d, as a partition between the lid part 12 and the power reception unit 13 and informing unit 14 is provided on the upper surface of the lower side member 11c. The lower side member 11c has a liquid circulation passage 11c2 that is provided so as to extend from the bottom side toward the outer circumference side of the recessed section 11a and through which the liquid stored in the recessed section 11a circulates. One end of the catheter is connected to the liquid circulation passage 11c2. As depicted in FIG. 3, protruding engagement sections 11c3 are formed on the outer circumference side of the partition wall 11c1 on the upper surface of the lower side member 11c, the protruding engagement sections 11c3 serving as a plurality of positioning structures for positioning the circuit board (described hereinafter) of the power reception unit 13 with respect to the main body 11 by being engaged by the circuit board. The plurality of protruding engagement sections 11c3 are arranged at intervals along the outer circumference side of the recessed section 11a. The recessed section 11a may also be referred to as, for example, a medical solution tank.

The lid part 12 is formed from a soft member, e.g., silicone rubber, through which an injection needle can be repeatedly inserted. The lid part 12 has a column-shaped lid body 12a, and a flange section 12b provided circumferentially entirely on the outer circumference portion of the lid body 12a and extending outward. The lid body 12a is a portion through which an injection needle is repeatedly inserted from the outside of the living body. The flange section 12b is a portion situated in the space 11d of the main body 11.

The power reception unit 13 has a circuit board 13a to which the informing unit 14 is connected, and a power reception coil 13b that is provided in the circuit board 13a and receives power from the power transmission coil (described hereinafter) of the outside-of-living-body unit 20.

As depicted in FIGS. 1 and 4, the circuit board 13a has, in the thickness direction, a plurality of conductor layers 13a2 provided by stacking, in an alternating pattern, insulator layers 13a1 formed from a resin material and conductor layers 13a2 formed from, for example, copper foil. For example, the circuit board 13a has 6 to 14 conductor layers 13a2. The circuit board 13a is formed in such a manner as to have a thickness size of, for example, 1.5 mm, has an inner diameter size larger than the outer diameter size of the partition wall 11c1 of the main body 11, and is formed in a circular-ring shape to surround the outer circumference side of the recessed section 11a of the main body 11. Meanwhile, as depicted in FIG. 3, recessed engagement sections 13c are formed in the circuit board 13a in such a manner as to be arranged at intervals circumferentially on the outer circumference side, the recessed engagement sections 13c serving as a plurality of positioning structures for positioning the circuit board 13a with respect to the lower side member 11c by engaging the protruding engagement sections 11c3 of the lower side member 11c. Furthermore, in addition to the power reception coil 13b, the circuit board 13a has formed thereat a resonance circuit (not illustrated) to which the power reception coil 13b is connected. A plurality of light-emitting units (described hereinafter) of the informing unit 14, which is connected to the resonance circuit and emits light by means of power received by the power reception coil 13b, are installed on the upper surface of the circuit board 13a.

As depicted in FIGS. 4 and 5, the power reception coil 13b is formed in the circuit board 13a by connecting conductor layers 13a2 adjacent to each other in the thickness direction of the circuit board 13a via vias 13a3. For example, the vias 13a3 are through vias, blind vias, or buried vias. For example, a conductor for forming the power reception coil 13b is formed so as to have a width size of 0.1 mm or greater. When through vias are used as the vias 13a3, conductor layers 13a2 adjacent to each other in the thickness direction do not need to be connected, conductor layers 13a2 having one or more conductor layers 13a2 interposed therebetween may be connected to each other. In particular, the power reception coil 13b is formed in the circuit board 13a by connecting the plurality of conductor layers 13a2 of the circuit board 13a to each other.

The vias 13a3 for forming the power reception coil 13b may be, for example, blind vias, buried vias, or through vias. The manufacturing cost can be reduced using through vias. Characteristics can be improved using buried vias.

The conductor for forming the power reception coil 13b preferably has a small width size in order to make the power reception coil 13b small-sized, but preferably has a large width size in order to suppress heat generation caused by a current increase. The conductor for forming the power reception coil 13b is typically set to have a width size of 1 mm and achieve one ampere of current. However, the present invention is not limited to this, and a comprehensive judgement may be made so as to set a width size.

The power reception coil 13b is not limited to a circular-ring shape, as long as the plurality of conductor layers 13a2 connected by the vias 13a3 are formed in a ring shape. For example, the power reception coil 13b may have an elliptical-ring shape, a polygonal-ring shape such as a rectangular-ring shape, or a ring shape having a combination of an arc and a straight line. In a case where the power reception coil 13b is disposed on the lower surface of the main body 11, the entirety of the circuit board 13a may be formed in a flat plate shape when the plurality of conductor layers 13a2 connected by the vias 13a3 are formed in a ring shape.

The power reception coil 13b preferably has a small number of conductor layers 13a2 in order to reduce the manufacturing cost, but preferably has a large number of conductor layers 13a2 in order to increase inductance. The power reception coil 13b in the present embodiment is set such that the number of conductor layers 13a2 falls within the range from 6 to 14.

The conductor of each of the plurality of conductor layers 13a2 of the power reception coil 13b may extend so as to assume a ring shape while forming one loop and connect to the conductor of an adjacent conductor layer 13a2, or the conductor of each of the plurality of conductor layers 13a2 may extend so as to assume a ring shape while forming two or more loops and connect to the conductor of an adjacent conductor layer 13a2.

The informing unit 14 is formed from a plurality of light-emitting units 14a. For example, the plurality of light-emitting units 14a are each a light emitting diode (LED), which emits light by having a voltage applied thereto, and, as depicted in FIG. 3, are arranged at intervals circumferentially on the upper surface of the circuit board 13a of the power reception unit 13. An emission direction in which each of the plurality of light-emitting units 14a emits light is an upward direction.

As depicted in FIG. 1, the outside-of-living-body unit 20 includes a housing 21 having such a size that a person can grip the same by hand, and a power transmission unit 22 provided in the housing 21. For example, the power transmission unit 22 has a power transmission coil 22a formed in a ring shape by repeatedly winding a conducting wire of copper, and a resonance circuit (not illustrated) to which the power transmission coil 22a is connected. Bringing the outside-of-living-body unit 20 close to the medical instrument 10 causes resonance to occur between the power transmission coil 22a of the power transmission unit 22 and the power reception coil 13b of the medical instrument 10, thereby transmitting power from the power transmission coil 22a to the power reception coil 13b.

In the medical device 1 configured as described above, the medical instrument 10 is used in a state of being implanted in a living body. A liquid such as a medical solution to be administered into the living body is injected into the recessed section 11a of the main body 11 via an injection needle such as a Huber-pointed needle inserted through the lid part 12 from the outside of the living body. The liquid injected into the recessed section 11a of the main body 11 will pass through the liquid circulation passage 11c2, circulate through a catheter, and be administered into a central vein of the living body.

For the medical instrument 10 implanted in the living body, the position of the lid part 12 in the living body needs to be identified when the injection needle is inserted through the lid part 12. Thus, when inserting the injection needle through the lid part 12 of the medical instrument 10 implanted in the living body, the outside-of-living-body unit 20 is brought, from the outside of the living body, close to the portion in which the medical instrument 10 is implanted, and electricity is caused to flow through the power transmission coil 22a. As a result, the power transmission coil 22a of the outside-of-living-body unit 20 is brought close to the power reception coil 13b of the medical instrument 10 such that power is transmitted from the power transmission coil 22a to the power reception coil 13b, and the plurality of light-emitting units 14a emit light by means of the power received by the power reception coil 13b. The plurality of light-emitting units 14a each emit light toward an area above the main body 11 such that light is emitted upward from the outer circumference side of the lid part 12, and thus the light arrives at the outer circumference portion of the lid part 12 at the surface of the living body, thereby allowing the position of the lid part 12 to be identified. Under this condition, the injection needle can be reliably inserted through the lid part 12 by inserting the injection needle with a portion surrounded by the plurality of light-emitting units 14a as the target.

As described above, according to the medical instrument of the present embodiment, a medical instrument 10 to be implanted in a living body includes a main body 11 that has a recessed section 11a extending in the thickness direction and having an upper surface with an opening and that is capable of storing a liquid in the recessed section 11a, a lid part 12 that is formed from a soft member through which an injection needle can be inserted and that closes the opening of the recessed section 11a, a power reception unit 13 that has a power reception coil 13b for receiving power sent from a power transmission coil 22a, and an informing unit 14 that is connected to the power reception unit 13 and informs information pertaining to the position of the lid part 12 by means of the power received by the power reception coil 13b, wherein the power reception unit 13 has a circuit board 13a in which a plurality of conductor layers 13a2 are formed in the thickness direction, and the power reception coil 13b is formed in the circuit board 13a by connecting the plurality of conductor layers 13a2 of the circuit board 13a to each other.

According to the medical device of the present embodiment, a medical device 1 includes an outside-of-living-body unit 20 that has a power transmission coil 22a for sending power and a medical instrument 10 that has a power reception coil 13b for receiving the power sent from the outside-of-living-body unit 20 and that is to be implanted in a living body, wherein the medical instrument 10 includes a main body 11 that has a recessed section 11a extending in the thickness direction and having an upper surface with an opening and that is capable of storing a liquid in the recessed section 11a, a lid part 12 that is formed from a soft member through which an injection needle can be inserted and that closes the opening of the recessed section 11a, a power reception unit 13 that has the power reception coil 13b, and an informing unit 14 that is connected to the power reception unit 13 and informs information pertaining to the position of the lid part 12 by means of the power received by the power reception coil 13b, the power reception unit 13 has a circuit board 13a in which a plurality of conductor layers 13a2 are formed in the thickness direction, and the power reception coil 13b is formed in the circuit board 13a by connecting the plurality of conductor layers 13a2 of the circuit board 13a to each other.

Accordingly, since the power reception coil 13b is formed in the circuit board 13a, the installation space for the power reception coil 13b can be reduced in comparison to when the power reception coil is formed by winding a conducting wire, so that the main body 11 can have a reduced size. Meanwhile, an adhesive is unnecessary when the circuit board and the power reception coil are integrally formed, in comparison to when the power reception coil is formed by winding a conducting wire. Furthermore, the power reception coil 13b and the circuit board 13a can be a single component, and the man-hours in the assembling work can be reduced. A large proportion of the power reception coil 13b is situated inside the circuit board 13a, so that the conductor for forming the power reception coil 13b can be small in width, and an occurrence of, for example, insulation breakdown and electric leakage can be suppressed. In comparison to when the power reception coil is formed by winding a conducting wire, the power reception coil 13b has a large surface area, so that the heat dissipation effect can be improved, and the influence of the skin effect of an alternating current can be suppressed. Meanwhile, the heat dissipation effect can be improved by increasing the thickness of a portion of the conductor for forming the power reception coil 13b.

The power reception coil 13b is formed integrally with the circuit board 13a, so forming the circuit board 13a integrally with the main body 11 or the lid part 12 eliminates the need for a dedicated space for accommodating the power reception unit 13, so that the main body 11 can have enhanced strength and a reduced size. Furthermore, a working process of connecting the circuit board 13a and the power reception coil 13b is unnecessary so that the manufacturing cost can be reduced.

The circuit board 13a is preferably formed in a ring shape surrounding the outer circumference side of the recessed section 11a of the main body 11.

In this way, the installation space for the circuit board 13a in the main body 11 can be reduced so that the main body 11 can have a further reduced size.

A positioning structure is preferably provided that positions the circuit board 13a with respect to the main body 11.

In this way, the circuit board 13a is positioned with respect to the main body 11 by attaching the circuit board 13a to the main body 11, so that the man-hours in the assembling work can be reduced.

In the present embodiment, a plurality of recessed engagement sections 13c formed at intervals circumferentially on the outer circumference side of the circuit board 13a are indicated as positioning structures. However, the present invention is not limited to this. In a positioning mechanism, a protruding engagement section projecting from the outer circumference side of the circuit board may position the circuit board with respect to the main body. In a positioning structure, the circuit board may be formed in an elliptical-ring shape so as to position the circuit board with respect to the main body.

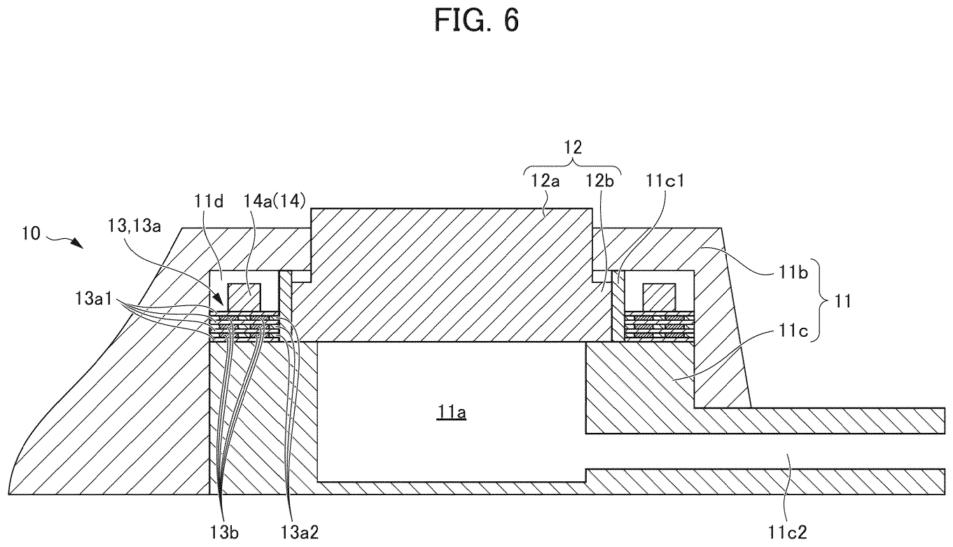

FIG. 6 illustrates another embodiment of the present invention and is a cross-sectional side view of a medical instrument. Component parts identical to those in the embodiment noted above are given like reference marks.

In the medical instrument 10 of the present embodiment, the number of windings of the power reception coil 13b is two or greater per one conductor layer 13a2 of the circuit board 13a.

Accordingly, according to the medical instrument and the medical device of the present embodiment, as in the embodiment noted above, the power reception coil 13b is formed in the circuit board 13a, thereby allowing the installation space for the power reception coil 13b to be reduced in comparison to when the power reception coil is formed by winding a conducting wire, so that the main body 11 can have a reduced size.

The number of windings of the power reception coil 13b is preferably two or greater per one conductor layer 13a2 of the circuit board 13a.

In this way, the number of conductor layers 13a2 to be used to form the power reception coil 13b having a required number of windings can be reduced, so that the thickness-direction size of the circuit board 13a can be reduced, and the main body 11 can have a reduced size in the thickness direction.

When the number of windings of the circuit board 13a per one conductor layer 13a2 is two or greater, the conductor layers 13a2 in the circuit board 13a can have a reduced size in the stacking direction, and the circuit board 13a can have a reduced size in the radial direction in comparison to when the circuit board and the power reception coil are separate components. For example, while a power reception coil formed from a copper wire having an outer diameter of 0.2 mm and a number of windings of 10 will have a ring thickness of 2 mm or greater, the power reception coil formed from the conductor layers 13a2 has a ring thickness of 1 to 1.5 mm.

In the embodiment noted above, the protruding engagement sections 11c3 provided on the lower side member 11c of the main body 11 and the recessed engagement sections 13c provided in the circuit board 13a engage each other, thereby forming positioning structures for positioning the circuit board 13a with respect to the lower side member 11c of the main body 11. However, the present invention is not limited to this. For example, a positioning structure for positioning the circuit board with respect to the main body may be provided such that: the outer circumference portion of the circuit board formed in a circular-ring shape is partially cut so as to form a straight edge surface; and the straight edge surface engages the main body. Alternatively, a positioning structure may be provided such that, for example: the inner circumferential portion of the circuit board having a ring shape has an elliptical shape or a polygonal shape; and the outer circumference portion of the partition wall of the main body has an elliptical shape or a polygonal shape with which the circuit board can be fitted.

In the embodiment noted above, the circuit board 13b is formed in a circular-ring shape. However, the present invention is not limited to this, and the circuit board may be formed in a polygonal-ring shape or a ring shape having a combination of a straight line and an arc. Arranging the power reception coil so as to assume a ring shape eliminates the need to form the circuit board in a ring shape; and for example, a circuit board shaped like a rectangular plate may be disposed below the recessed section of the main body, and a power reception coil may be formed in a ring shape in the circuit board shaped like a rectangular plate.

In the embodiment noted above, the plurality of light-emitting units 14a that emit light by means of power received by the power reception coil 13b are indicated as the informing unit. However, the present invention is not limited to this. For example, a buzzer or speaker that emits a sound by means of power received by the power reception coil may be used as the informing unit, as long as the same informs information pertaining to the position of the lid part. In the embodiment noted above, an LED is indicated as the light-emitting unit 14a. However, the light-emitting unit 14a is not limited to an LED, as long as the same emits light by means of power received by the power reception coil 13b. In the embodiment noted above, a plurality of light-emitting units 14a are arranged at intervals on the outer circumference side of the lid part 12 so that the position of the lid part 12 can be identified. However, the present invention is not limited to this. For example, one light-emitting unit that emits light assuming a ring shape to surround the outer circumference side of the lid part may be used, as long as the same allows the position of the lid part to be identified.

In the embodiment noted above, power is transmitted from the outside-of-living-body unit 20 to the medical instrument 10 by means of so-called magnetic resonance produced by a resonance circuit formed at the circuit board 14. However, power may be transmitted by electromagnetic induction.

In this regard, a power reception circuit formed in the medical instrument receives AC power irrespective of whether power is transmitted by electromagnetic induction or by magnetic resonance. In order to allow LEDs to emit light efficiently by means of power received from the power transmission coil, a full-wave rectifying circuit may be formed in the power reception circuit so as to convert the power into a direct current by full-wave rectification, or a plurality of LEDs having a rectification effect may be connected in parallel to the power reception circuit such that some of the plurality of LEDs are connected in the forward direction of the power reception circuit while the other LEDs are connected in the backward direction.

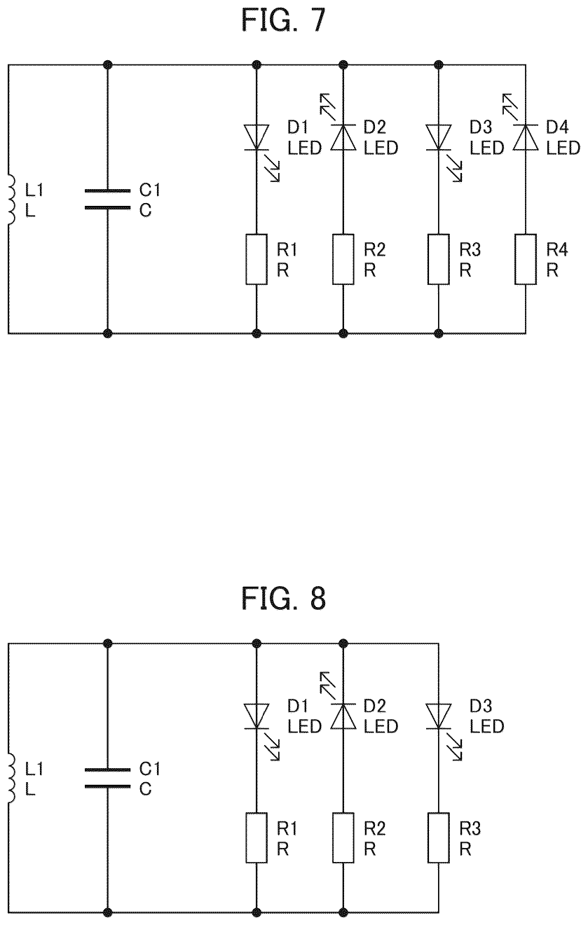

When an even number of LEDs are connected to the power reception circuit, the LEDs can be equal in brightness by making it so that, as depicted in FIG. 7, the number of LEDs connected in the forward direction of the power reception circuit is equal to the number of LEDs connected in the backward direction. In this way, the power reception circuit can be formed from fewer components, thereby reducing the manufacturing cost. Meanwhile, the brightness of some of the LEDs can be changed by adjusting the magnitude of a resistor to be installed in the power reception circuit.

In a case where an odd number of LEDs are connected to the power reception circuit, when the plurality of LEDs do not need to be the same in brightness, the number of LEDs connected in the forward direction of the power reception circuit may be different from that in the backward direction thereof, as depicted in FIG. 8.

Causing a plurality of LEDs connected to the power reception circuit to each emit light having a different color allows an injection needle to be intentionally inserted into various locations on the lid part 12, so that the lid part 12 can have an extended service life. For example, some of the plurality of LEDs connected to the power reception circuit are caused to emit light having a wavelength of approximately 650 nm (red), and the others are caused to emit light having a wavelength of approximately 600 nm (yellow).

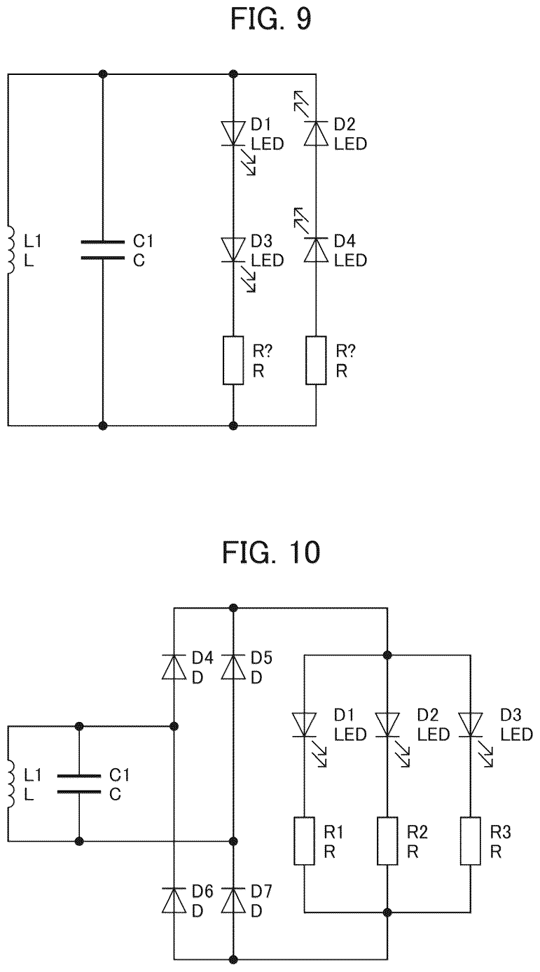

The plurality of LEDs may be connected in parallel to the power reception circuit as depicted in FIGS. 7 and 8, or may be connected in series thereto as depicted in FIG. 9. However, the plurality of LEDs are preferably connected in parallel to the power reception circuit, because a high voltage would be required to emit light if the same are connected in series to the power reception circuit.

In a case where an odd number of LEDs are connected to the power reception circuit, in order to make the plurality of LEDs equal in brightness, a full-wave rectifying circuit may be formed by arranging, as depicted in FIG. 10, four diodes in a bridge formation in the power reception circuit. In this situation, diodes having a low ON voltage, such as Schottky barrier diodes, are preferably used.

The power reception coil 13 and the circuit board 14 may be surface-treated with a coating material. It is preferable that the coating material used in this case is transparent with respect to visible light and has high biocompatibility.

Since the power reception coil 13b is formed integrally with the circuit board 13a, an adhesive for connecting the circuit board and the power reception coil to each other is unnecessary, so that the influence that the adhesive would impose on a living body can be eliminated. Forming the power reception coil 13b integrally with the circuit board 13a allows the strength of the medical instrument to be enhanced so that the reliability of the medical instrument can be improved, and the handling of the medical instrument is facilitated. Furthermore, since a large proportion of the power reception coil 13b is disposed inside the circuit board 13a, an occurrence of, for example, insulation breakdown and electric leakage can be suppressed. Meanwhile, the conductor for forming the power reception coil 13b may have a reduced width size so that an occurrence of break in the conductor can be suppressed.

### EXPLANATION OF REFERENCE NUMERALS

1: Medical device
10: Medical instrument
11: Main body
11a: Recessed section
12: Lid part
13: Power reception unit
13a: Circuit board
13a2: Conductor layer
13b: Power reception coil
14: Informing unit
20: Outside-of-living-body unit
22a: Power transmission coil

1. A medical instrument to be implanted in a living body, the medical instrument comprising:
a main body that has a recessed section extending in a thickness direction and having an upper surface with an opening and that is capable of storing a liquid in the recessed section;
a lid part that is formed from a soft member through which an injection needle can be inserted and that closes the opening of the recessed section;
a power reception unit that has a power reception coil for receiving power sent from a power transmission coil; and
an informing unit that is connected to the power reception unit and informs information pertaining to a position of the lid part by means of the power received by the power reception coil, wherein
the power reception unit has a circuit board in which a plurality of conductor layers are formed in the thickness direction, and
the power reception coil is formed in the circuit board by connecting the plurality of conductor layers of the circuit board to each other.

2. The medical instrument according to claim 1, wherein
the circuit board is formed in a ring shape surrounding an outer circumference side of the recessed section of the main body.

3. The medical instrument according to claim 2, the medical instrument comprising:
a positioning structure that positions the circuit board with respect to the main body.

4. The medical instrument according to claim 1, wherein
a number of windings of the power reception coil is two or greater per one conductor layer of the circuit board.

5. A medical device comprising an outside-of-living-body unit that has a power transmission coil for sending power and a medical instrument that has a power reception coil for receiving the power sent from the outside-of-living-body unit and that is to be implanted in a living body, wherein
the medical instrument includes
a main body that has a recessed section extending in a thickness direction and having an upper surface with an opening and that is capable of storing a liquid in the recessed section,
a lid part that is formed from a soft member through which an injection needle can be inserted and that closes the opening of the recessed section,
a power reception unit that has the power reception coil, and
an informing unit that is connected to the power reception unit and informs information pertaining to a position of the lid part by means of the power received by the power reception coil,
the power reception unit has a circuit board in which a plurality of conductor layers are formed in the thickness direction, and
the power reception coil is formed in the circuit board by connecting the plurality of conductor layers of the circuit board to each other.
